(19) United States
(12) Patent Application Publication
(10) Pub. No.: US 2021/0134394 A1
(43) Pub. Date: May 6, 2021

(54) ENDPOINT ANALYSIS IN EARLY CANCER DETECTION

(71) Applicant: GRAIL, INC., Menlo Park, CA (US)

(72) Inventors: Peter D. Freese, San Francisco, CA (US); Collin Melton, Menlo Park, CA (US); Earl Hubbell, Palo Alto, CA (US)

(21) Appl. No.: 17/066,821

(22) Filed: Oct. 9, 2020

Related U.S. Application Data
(60) Provisional application No. 62/922,223, filed on Oct. 11, 2019.

(51) Int. Cl. G16B 40/00; G16H 50/20; G06N 3/08

(52) U.S. Cl. CPC G16B 40/00; G06N 3/08; G16H 50/20

(57) ABSTRACT

A system and method for determining a presence of cancer in a test sample from a test subject comprising a set of fragments of deoxyribonucleic acid (DNA) is described. Locations along a genome of the test subject that are predictively significant in cancer detection may be identified through probabilistic analyses based on a comparison of the count of non-cancer fragments expected to terminate at a location and a count of fragments observed to terminate at the location. Based on the comparison, a p-value for each location is determined and is compared to a p-value threshold to determine predictively significant genomic locations, and a classifier is trained based on these locations. The system inputs a test feature vector containing counts of endpoint fragments from a test sample to the classifier, which generates a cancer prediction describing a likelihood the test sample has cancer and/or is of a particular cancer type.

### BACKGROUND

### Field of Art

Cell-free DNA (cfDNA) molecules are present in circulating plasma, urine, and other body fluids of humans, resulting from certain cellular processes, such as apoptosis. Analysis of endpoints for cfDNA is increasingly recognized as a valuable diagnostic tool for the detection, diagnosis, and/or monitoring of cancer. For example, specific patterns of cfDNA fragment endpoints may be useful as markers for non-invasive diagnostics using circulating cfDNA. However, there remains a need in the art for improved methods for analyzing cfDNA fragment endpoints for the detection, diagnosis, and/or monitoring of diseases, such as cancer.

### SUMMARY

Early detection of cancer in subjects is important as it allows for earlier treatment and therefore a greater chance for survival. Endpoint analysis of DNA fragments in a cell-free DNA (cfDNA) sample provides insight into whether a subject may have cancer, and further insight into how advanced the cancer is and what type of cancer the subject may have. Towards that end, this description includes systems and methods for analyzing endpoints of cfDNA fragments to determine a subject's likelihood of having cancer.

An analytics system processes a multitude of cfDNA fragments from one or more cancer samples and a set of fragments from one or more non-cancer samples. As described herein, a cfDNA fragment originates at a start point and terminates at an end point, both of which are aligned to a reference genome. The techniques described herein, collectively referred to as endpoint analysis, may be applied to determine statistically significant positions of start points, end points, or both. Accordingly, as described herein, the term “endpoint” may be used generally to refer to either “start points” or “end points” of a cfDNA fragment.

The analytics system creates a cancer endpoint position vector containing counts of DNA fragments from cancer samples with an endpoint at each location within a genome. Depending on the implementation, the evaluated endpoints may be fragment start points or fragment end points. As described herein, each entry of an endpoint position vector corresponds to a location on a subject's chromosome, also referred to as genomic location. Similarly, the analytics system creates a non-cancer endpoint position vector containing entries describing counts of DNA fragments from non-cancer samples with an endpoint at each genomic location. Based on a comparison of counts of cancer endpoints to non-cancer endpoints at each location, the analytics system determines whether each genomic location is predictively significant. The predictive significance of a genomic location characterizes whether a cfDNA fragment with an endpoint at the genomic location provides significant insight into determining a subject's likelihood of having a cancer.

Using fragments of a test sample, the analytics system trains and deploys a cancer classifier to generate a cancer prediction for the test sample. With regards to the predictively significant locations, the analytics system generates an endpoint position vector for the test sample in a similar manner to those cfDNA fragments described above. From the test sample, the analytics system isolates a plurality of cfDNA fragments and determines a count of fragments with endpoints at each predictively significant location. The analytics system encodes the count of endpoints at each location as a feature in a feature vector that is input to a classifier, and returns a cancer prediction. As described herein, such a cancer classifier may perform operations including, but not limited to, a logistic regression, a multinomial regression, and a non-linear regression.

Regarding which training samples are used to train the cancer classifier, the analytics system uses training samples that have already been labeled as having one or more cancer types, as well as training samples from healthy individuals that are labeled as non-cancer. Each training sample includes a set of cfDNA fragments with a start point and an end point at a genomic location on a subject's chromosome. For each training sample, the analytics system generates a feature vector that includes feature values representing a count of fragment endpoints at each predictively significant genomic location. The analytics system may group the training samples into sets of one or more training samples for iterative training of the cancer classifier. The analytics system inputs each set of feature vectors into the cancer classifier and adjusts classification parameters in the cancer classifier such that a function of the cancer classifier calculates cancer predictions that accurately predict the labels of the training samples in the set. The cancer classifier iterates the above steps through each set of training samples during training. In one embodiment, each training sample includes a set of cfDNA fragments and a count of endpoints at predictively significant locations on the genome.

During deployment, the analytics system generates a feature vector (i.e., an endpoint position vector) for a test sample in a similar manner to the training samples, i.e., by determining count of fragment endpoints for each genomic location. The analytics system inputs the position vector for the test sample into the cancer classifier which returns a cancer prediction. In one embodiment, the cancer classifier may be configured as a binary classifier to return a likelihood that a subject has or does not have cancer. In another embodiment, the cancer classifier may be configured as a multiclass classifier to return a prediction with prediction values for certain cancer types and/or stages being categorized.

Examples of cancer types which may be predicted using the cancer classifier include a breast cancer type, a colorectal cancer type, an esophageal cancer type, a head/neck cancer type, a hepatobiliary cancer type, a lung cancer type, a lymphoma cancer type, an ovarian cancer type, a pancreas cancer type, an anorectal cancer type, a cervical cancer type, a gastric cancer type, a leukemia cancer type, a multiple myeloma cancer type, a prostate cancer type, a renal cancer type, a thyroid cancer type, a uterine cancer type, a brain cancer type, a sarcoma cancer type, a neuroendocrine cancer type.

In some embodiments, a method for classifying cancer and a system comprising a hardware processor for performing steps of the method are disclosed. The method comprises: accessing a first set of training data and a second set of training data, the first set of training data indicating fragment endpoints of cell-free fragments from cancer samples and the second set of training data indicating fragment endpoints of cell-free fragments from non-cancer samples; generating a first vector representative of the first set of training data and a second vector representative of the second set of training data, wherein each entry within the first vector and each entry within the second vector includes a count of cell-free fragments from the cancer samples and from the non-cancer samples, respectively, having an endpoint at a particular genomic location; computing, for each genomic location, an associated p-value representative of a significance of the entry corresponding to the genomic location within the first vector relative to the entry corresponding to the genomic location within the second vector; identifying a set of genomic locations associated with p-values less than a p-value threshold; training a classifier based on the identified set of genomic locations; and classifying a test sample as a cancer sample or a non-cancer sample by: determining counts of cell-free fragments in the test sample having an endpoint at each of the identified genomic locations; and applying the classifier to the determined counts to determine if the test sample is a cancer sample or a non-cancer sample.

In one embodiment, computing a p-value for each genomic location comprises: determining a prior gamma distribution for the corresponding entry of the second vector, the prior gamma distribution parameterized by a prior shape parameter and a prior rate parameter used to compute a first mean of the prior gamma distribution and a first variance of the prior gamma distribution.

In one embodiment, computing a p-value for each genomic location further comprises: updating the prior gamma distribution for the corresponding entry of the second vector based on the count of endpoints within the corresponding entry of the second vector to produce a posterior distribution of expected counts within the entry of the second vector, the posterior distribution parameterized by a posterior shape parameter and a posterior rate parameter.

In one embodiment, computing a p-value for each genomic location further comprises: computing a second mean and a second variance of a negative binomial distribution for the entry of the first vector using the posterior shape parameter and the posterior rate parameter.

In one embodiment, computing the second mean and the second variance of the negative binomial distribution comprises scaling the posterior shape parameter and the posterior rate parameter based on a total count of cancer fragments from the cancer samples and a total count of non-cancer fragments from the non-cancer samples, and wherein the second mean and the second variance of the negative binomial distribution are computed based on the scaled posterior shape parameter and the scaled posterior rate parameter.

In one embodiment, computing a p-value for each genomic location further comprises: computing, using the negative binomial distribution, a probability that a count of endpoints within an entry of the first vector corresponding to the genomic location is expected given a count of endpoints within an entry of the second vector corresponding to the genomic location or exceeds the count of endpoints within the entry of the second vector, wherein the computed probability comprises the p-value for the genomic location.

In one embodiment, computing a p-value for each genomic location further comprises: computing, using the negative binomial probability mass function and for each integer greater than or equal to the count of endpoints within the entry of the first vector corresponding to the genomic location, a probability that the count of endpoints within the entry of the first vector corresponding to the genomic location is equal to the integer; and summing the computed probabilities to produce the p-value.

In one embodiment, the cancer samples and the non-cancer samples comprise cell-free fragments between 50 and 140 bp.

In one embodiment, the cancer samples are from subjects with a particular type of cancer, and wherein the classifier is configured to classify the test sample as a cancer sample with the particular type of cancer or a non-cancer sample.

In one embodiment, the classifier is a multiclass classifier, and is configured to classify the test sample as a sample associated with one of a plurality of types of cancer.

In one embodiment, the method further comprises: identifying a second set of genomic locations associated with p-values greater than the p-value threshold but less than a second p-value threshold; and training the classifier based additionally on the identified second set of genomic locations; wherein classifying the test sample further comprises determining counts of cell-free fragments in the test sample having an endpoint at each of the second set of genomic locations.

In one embodiment, the p-value threshold is less than 10⁻⁴.

In one embodiment, the p-value threshold is one of: 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, 10⁻¹⁸, 10⁻¹⁹, and 10⁻²⁰.

In one embodiment, the set of genomic locations comprise less than 2,000, less than 5,000, less than 10,000, less than 50,000, less than 100,000, less than 500,000, less than 1 million, or less than 5 million unique genomic locations.

In some embodiments, a method for determining whether a test subject has cancer and a system including a hardware processor for performing the method are disclosed. The method comprises: determining counts of cell-free fragments in a test sample having an endpoint at each of a plurality of genomic locations listed in Table I, Table II, Table III, or Table IV; assessing counts of cell-free fragments in cancer training samples having an endpoint at each of the plurality of genomic locations listed in Table I, Table II, Table III, or Table IV; assessing counts of cell-free fragments in non-cancer training samples having an endpoint at each of the plurality of genomic locations listed in Table I, Table II, Table III, or Table IV; and comparing the counts for the test sample, the counts for the cancer training samples, and the counts for the non-cancer training samples to determine if the test sample is cancer sample or non-cancer sample.

In one embodiment, the further comprises the preceding step of enriching cell-free fragments in a test sample having an endpoint at each of the plurality of genomic locations listed in Table I, Table II, Table III, or Table IV by using a plurality of enrichment oligonucleotides.

In one embodiment, the plurality of genomic locations comprises at least 50 genomic locations listed in Table I, Table II, Table III, or Table IV.

In one embodiment, the plurality of genomic locations comprises at least 100 genomic locations listed in Table I, Table II, or Table III.

In one embodiment, the plurality of genomic locations comprises at least 300 genomic locations listed in Table I, or Table II.

In one embodiment, the plurality of genomic locations comprises at least 500 genomic locations listed in Table I.

In one embodiment, the plurality of genomic locations comprises all of genomic locations listed in Table I, Table II, Table III, or Table IV.

In some embodiments, an assay panel for enriching polynucleotides from a cell-free DNA sample is disclosed. The assay panel comprises at a plurality of different polynucleotide probes, wherein each of the plurality of different polynucleotide probes is configured to hybridize to a nucleotide sequence of a polynucleotide having a genomic endpoint listed in Table I, Table II, Table III, or Table IV. A composition comprising DNA that has been processed to convert unmethylated cytosine to uracil and the assay panel is also disclosed.

In one embodiment, the plurality of different polynucleotide probes comprises at least 50 different polynucleotide probes each configured to hybridize to a nucleotide sequence of a polynucleotide having a genomic endpoint listed in Table I, Table II, Table III, or Table IV.

In one embodiment, the plurality of different polynucleotide probes comprises at least 100 different polynucleotide probes each configured to hybridize to a nucleotide sequence of a polynucleotide having a genomic endpoint listed in Table I, Table II, or Table III.

In one embodiment, the plurality of different polynucleotide probes comprises at least 300 different polynucleotide probes each configured to hybridize to a nucleotide sequence of a polynucleotide having a genomic endpoint listed in Table I, or Table II.

In one embodiment, the plurality of different polynucleotide probes comprises at least 500 different polynucleotide probes each configured to hybridize to a nucleotide sequence of a polynucleotide having a genomic endpoint listed in Table I, or Table II.

In one embodiment, the plurality of different polynucleotide probes comprises at least 1000 different polynucleotide probes each configured to hybridize to a nucleotide sequence of a polynucleotide having a genomic endpoint listed in Table I.

In one embodiment, each of the at least 50 different polynucleotide probes is conjugated to an affinity moiety.

In one embodiment, the affinity moiety is a biotin moiety.

In one embodiment, each of the plurality of different polynucleotide probes comprises at least 75 nucleotides.

In one embodiment, the plurality of different polynucleotide probes comprise at least 50 different pairs of polynucleotide probes, wherein each pair of the at least 50 different pairs of probes comprises two different probes configured to overlap with each other by an overlapping sequence of at least 30 or more contiguous nucleotides and wherein each of the two different probes of each of the pair of probes is configured to hybridize to a nucleotide sequence of a polynucleotide having a genomic endpoint listed in Table I, Table II, Table III, or Table IV.

In one embodiment, the plurality of different polynucleotide probes comprise at least 100 different pairs of polynucleotide probes, wherein each pair of the at least 100 different pairs of probes comprises two different probes configured to overlap with each other by an overlapping sequence of at least 30 or more contiguous nucleotides and wherein each of the two different probes of each of the pair of probes is configured to hybridize to a nucleotide sequence of a polynucleotide having a genomic endpoint listed in Table I, Table II, or Table III.

In one embodiment, the plurality of different polynucleotide probes comprise at least 50 different pairs of polynucleotide probes, wherein each pair of the at least 300 different pairs of probes comprises two different probes configured to overlap with each other by an overlapping sequence of at least 30 or more contiguous nucleotides and wherein each of the two different probes of each of the pair of probes is configured to hybridize to a nucleotide sequence of a polynucleotide having a genomic endpoint listed in Table I, or Table II.

In one embodiment, the plurality of different polynucleotide probes comprise at least 500 different pairs of polynucleotide probes, wherein each pair of the at least 500 different pairs of probes comprises two different probes configured to overlap with each other by an overlapping sequence of at least 30 or more contiguous nucleotides and wherein each of the two different probes of each of the pair of probes is configured to hybridize to a nucleotide sequence of a polynucleotide having a genomic endpoint listed in Table I, or Table II.

In one embodiment, the plurality of different polynucleotide probes comprise at least 1000 different pairs of polynucleotide probes, wherein each pair of the at least 1000 different pairs of probes comprises two different probes configured to overlap with each other by an overlapping sequence of at least 30 or more contiguous nucleotides and wherein each of the two different probes of each of the pair of probes is configured to hybridize to a nucleotide sequence of a polynucleotide having a genomic endpoint listed in Table I.

In some embodiments, a method for detecting cancer and a system including a hardware processor for performing the method are disclosed. The method comprises: (a) obtaining a cell-free DNA test sample from a subject suspected of having cancer, the test sample comprising a plurality of cell-free DNA molecules; (b) combining the cell-free DNA molecules with the assay panel of any one of claims 22-37 and enriching for a subset of cell-free DNA molecules having a genomic endpoint listed in Table I, Table II, Table III, or Table IV; (c) sequencing the subset of the cell-free DNA molecules, thereby obtaining a set of sequence reads; and (d) applying the set of sequence reads or a test feature vector derived from the set of sequence reads to a model trained on a cancer set of converted DNA sequences from a plurality of training subjects with cancer and a non-cancer set of converted DNA sequences from a plurality of training subjects without cancer, wherein both the cancer set of converted DNA sequences and the non-cancer set of converted DNA sequences comprise a plurality of training fragments.

In one embodiment, the model comprises a kernel logistic regression classifier, a random forest classifier, a mixture model, a convolutional neural network, or an autoencoder model.

In one embodiment, the method further comprises determining a cancer classification by evaluating the set of sequence reads, wherein the cancer classification is a presence or absence of cancer, a type of cancer, and/or a stage of cancer.

In one embodiment, the plurality of training subjects with cancer comprises training subjects with at least 3 different types of cancer.

In one embodiment, the at least 3 different types of cancer are selected from blood cancer, breast cancer, colorectal cancer, esophageal cancer, head and neck cancer, hepatobiliary cancer, lung cancer, ovarian cancer, and pancreatic cancer.

### BRIEF DESCRIPTION OF DRAWINGS

FIG. 1 is a flowchart describing a process for generating endpoint position vectors for samples of cancer cfDNA fragments and non-cancer cfDNA fragments, according to an embodiment.

FIG. 2A is a flowchart describing a process for identifying predictively significant genomic locations, according to one embodiment.

FIG. 2B illustrates an example identification of predictively significant genomic locations, according to one embodiment.

FIG. 3 is a flowchart describing a process for identifying predictively significant genomic locations using statistical analyses, according to one embodiment.

FIG. 4A illustrates a flowchart of devices for sequencing nucleic acid samples according to one embodiment.

FIG. 4B is a block diagram of an analytics system, according to an embodiment.

FIG. 5 is a flowchart describing a process of training a cancer classifier, according to an embodiment.

FIGS. 6-7 illustrate graphs showing a sensitivity by stage analysis of a cancer classifier, according to one embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

### I.A. Overview of Endpoint Analysis

In accordance with the present description, cfDNA fragments from a sample are sequenced to identify the genomic location at which each fragment terminates. Alternatively, cfDNA fragments from a sample may be sequenced to identify the genomic location at which each fragment originates. Biologically, particular genomic locations may provide insight into whether or not a patient has cancer or has a particular type of cancer based on the number fragments that terminate, or alternatively originate, at the locations. Accordingly, statistical analyses of cfDNA fragments endpoints may provide a valuable tool in the detection of cancer in a subject. An elevated density of cfDNA fragment endpoints observed at a particular genomic location typically associated with a low endpoint density in healthy subjects may provide valuable diagnostic insight. Accordingly, the description herein discloses processes or techniques for identifying genomic locations known to be predictively significant in early cancer detection.

A test sample may be sequenced using known sequencing techniques to generate a library of cfDNA fragments aligned to a reference genome. Analysis of the count of endpoints observed at one or more previously determined predictively significant genomic locations on the reference genome may be used as inputs for a cancer classifier to identify the test sample as a non-cancer type or a cancer type. In some implementations, the classifier is trained to identify a particular type of cancer of a test sample based on the endpoint counts at one or more predictively significant genomic locations.

As described above, endpoints generally refer to both start points at which a fragment originates and end points at which a fragment terminates. For clarity, some implementations of the techniques and process for generating cancer predictions are described herein with reference to end points of DNA fragments. However, one skilled in the art would recognize that the same implementations or examples can be performed by analyzing start points of such DNA fragments. As an example, one or more predictively significant locations may be identified based on counts of fragment start points observed at locations along the reference genome (e.g., the genomic location at which each fragment originates). In such an implementation, a cancer classifier is trained to generate a cancer prediction using counts of fragment start points occurring at one or more predictively significant locations. Further, it should be noted that in addition to detecting cancer, the endpoint analysis described herein can be performed to train a classifier able to detect human conditions other than cancer (such as particular diseases, genetic conditions, and the like).

### I.B. Definitions

The term “individual” refers to a human individual. The term “healthy individual” refers to an individual presumed to not have a cancer or a disease. The term “subject” refers to an individual who is known to have, or potentially has, a cancer or disease.

The term “cell free nucleic acid” or “cfNA” refers to nucleic acid fragments that circulate in an individual's body (e.g., blood) and originate from one or more healthy cells and/or from one or more cancer cells. The term “cell free DNA,” or “cfDNA” refers to deoxyribonucleic acid fragments that circulate in an individual's body (e.g., blood). Additionally, cfNAs or cfDNA in an individual's body may come from other non-human sources.

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “sequence read” refers to a nucleotide sequence obtained from a nucleic acid molecule from a test sample from an individual. Sequence reads can be obtained through various methods known in the art.

The term “sequencing depth” or “depth” refers to a total number of sequence reads or read segments at a given genomic location or loci from a test sample from an individual.

The term “end point” refers to a genomic location at which a cfDNA fragment terminates and the term “start point” refers to a genomic location from which a cfDNA fragment originates. The term “endpoint” generally refers to both end points and start points of cfDNA fragments, for example, the nucleotide at the beginning or start location and/or the nucleotide at the end location in a cfDNA fragment. The term “statistically significant endpoint” refers to a genomic location which has been determined to provide insight in determining a subject's likelihood of having cancer.

### II. Sample Processing Using Endpoint Analysis

### II.A. Generating Endpoint Vectors for DNA Fragments

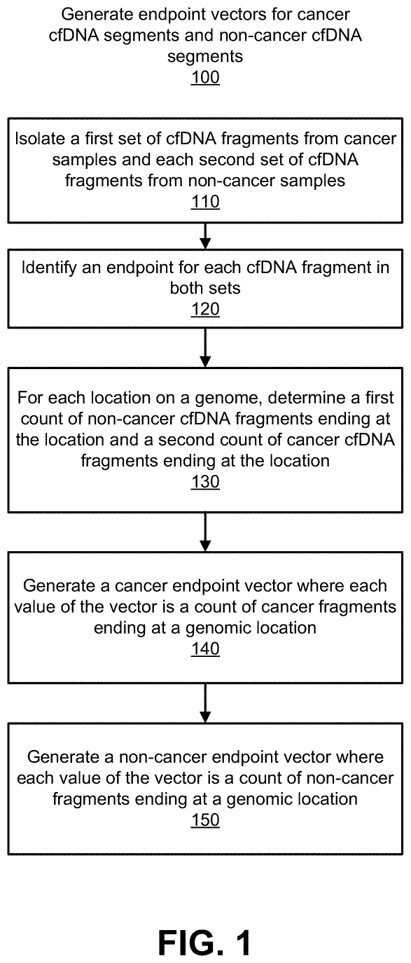

FIG. 1 is a flowchart describing a process for generating endpoint position vectors for a sample of cancer cfDNA fragments and non-cancer cfDNA fragments, according to an embodiment. To analyze DNA fragment endpoints, an analytics system first obtains samples from one or more individuals comprising a plurality of labeled cfDNA molecules. Generally, samples may be from healthy individuals, subjects known to have or suspected of having cancer, or subjects where no prior information is known. The sample may be a sample selected from the group consisting of blood, plasma, serum, urine, fecal, and saliva samples. Alternatively, the sample may be selected from the group consisting of whole blood, a blood fraction (e.g., white blood cells (WBCs)), a tissue biopsy, pleural fluid, pericardial fluid, cerebral spinal fluid, and peritoneal fluid. In additional embodiments, the process 100 may be applied to sequence other types of DNA molecules.

From each sample, the analytics system isolates 110 each cfDNA fragment and prepares a sequencing library. During the period in which the analytics system identifies predictively significant genomic locations, the analytics system receives samples that are labeled as either cancer-type or non-cancer type. Accordingly, the cfDNA fragments isolated from those samples are labeled in the sequencing library as “cancer-type” or “non-cancer type”. In comparison when implementing a cancer classifier trained to output a cancer prediction using endpoint analysis, the test samples to which the classifier is applied may not be labeled. Accordingly, the analytics system generates a sequencing library from the received test samples, but cfDNA fragments in the library may not be labeled.

Optionally, the sequencing library may be enriched for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified endpoints of interest to the researcher. Once prepared, the sequencing library or a portion thereof can be sequenced to obtain a plurality of sequence reads. The sequence reads may be in a computer-readable, digital format for processing and interpretation by computer software.

From the sequence reads, the analytics system identifies 120 an endpoint or endpoints for each cfDNA fragment based on an alignment to a reference genome (e.g., including the nucleotide at the start location and/or the nucleotide at the end location of the cfDNA fragment). The reference genome includes genomic locations along a chromosome for the subject from which the sample was obtained, for example a reference genome for a human would include approximately 3 billion base pairs, each of which is a genomic location on a human chromosome. For ease of analysis, the sequence library may be divided into a first section of sequence reads for cancer-type fragments and a second section of sequence reads for non-cancer-type fragments. For each location on the reference genome, the analytics system determines 130 a first count of cancer cfDNA fragments with endpoints at the location and a second count of non-cancer cfDNA fragments with endpoints at the location.

Based on the determined endpoint counts of cancer fragments, the analytics system generates 140 a cancer endpoint position vector. The vector comprises a number of entries equivalent to the number of locations on the reference genome, such that each entry corresponds to a distinct genomic location on the reference genome. For example, the feature value assigned to each entry may represent a count of cancer fragments with end points terminating at the corresponding genomic location. For example, if 3,000 cancer fragments have end points at genomic location 4 million bp, the entry in the cancer endpoint vector corresponding to genomic location 4 million bp is assigned a value of 3,000. In some implementations, the values assigned to entries in an endpoint position vector may be scaled by a constant factor to account for the sample's sequencing depth, for example an entry of 3,000 may be scaled by a factor of 1000 to 3. Consistent with the description above, the analytics system generates 150 a non-cancer endpoint position vector where each entry is assigned a value representing a count of non-cancer fragments with endpoints at a genomic location corresponding to the entry. Consistent with the description of the cancer endpoint position vector, the non-cancer endpoint position vector is generated based on the same reference genome such that both endpoint position vectors include the same number of entries and corresponding entries in each endpoint position vector are associated with the same genomic location of the reference genome.

### II.B. Identifying Predictively Significant Genomic Locations

The analytics system determines predictively significant genomic locations for a reference genome using a non-cancer endpoint position vector and a cancer endpoint position vector generated from a set of labeled samples. For each genomic location in the reference genome, the analytics system determines whether the location is a predictively significant location based on a comparison of the count of cancer fragments terminating at the location to the count of non-cancer fragments terminating at the location. In one embodiment, the analytics system computes a p-value for each genomic location representing a likelihood that the location is predictively significant in the detection of cancer. The process for computing a p-value will be further described below in section II.B.1. The analytics system may identify locations on the reference genome having p-values below a threshold as “predictively significant locations.”

In another embodiment, the analytics system may implement various other probabilistic models for determining predictively significant locations. Examples of other probabilistic models include a mixture model, a deep probabilistic model, a neural network, and the like. In some embodiments, the analytics system may use any combination of the processes described below for determining predictively significant locations. With the identified predictively significant locations, the analytics system may filter the set of endpoint position vector entries for a sample for use in other processes, e.g., for use in training and deploying a cancer classifier, as a feature vector for classification by a trained classifier, and the like.

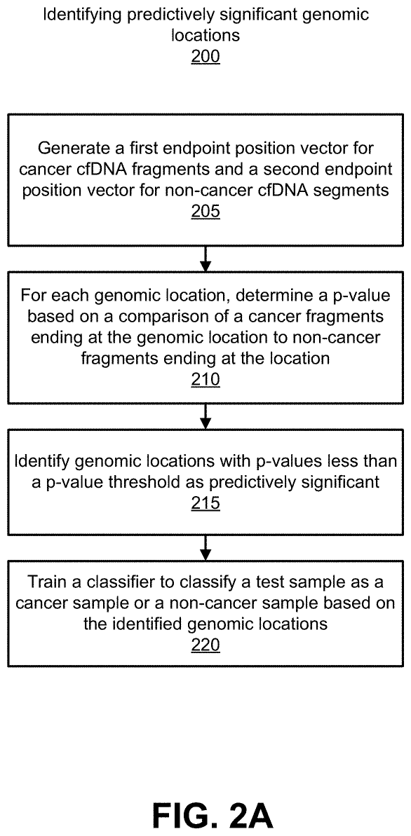

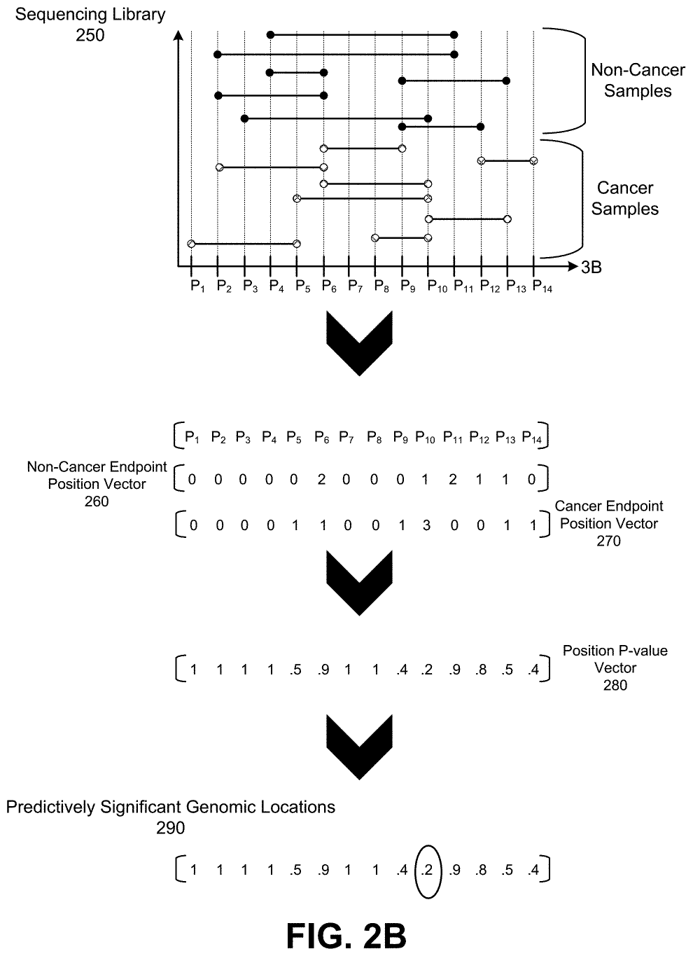

FIG. 2A is a flowchart describing a process for identifying predictively significant genomic locations, according to one embodiment. Consistent with the description above with reference to FIG. 1, the analytics system generates 205 a first endpoint position vector for cancer cfDNA fragments from one or more cancer samples and a second endpoint position vector for non-cancerous cfDNA fragments from one or more non-cancer samples. Each endpoint position vector is generated based on sequence reads aligned relative to a shared reference genome. Accordingly, for each genomic location, the analytics system determines 210 a p-value. The analytics system compares the count of cancer fragment end points at each genomic location to the count of non-cancer fragment end points at the location. In such an endpoint analysis, the count of non-cancer fragments with end points at each genomic location functions as a control group. In comparison, the count of cancer fragments terminating at each genomic location provides insight into whether the location is predictively significant for cancer diagnoses. The determined p-value for each location characterizes a likelihood that an abnormal count of cancer fragments terminating at the location relative to the number of non-cancer fragments terminating at the location is statistically significant enough to provide predictive insight into whether a subject has cancer.

The analytics system identifies 215 genomic locations with a p-value less than a p-value threshold as predictively significant. Because the identified locations provide additional insight regarding cancer detection in a subject, the analytics system trains 220 a classifier to classify a test sample as a cancer sample or a non-cancer sample. During the training of the classifier, a training dataset comprises a combination of labeled cancer samples and labeled non-cancer samples. Additionally, for each labeled non-cancer sample, the analytics system determines a count of non-cancer fragments terminating at each predictively significant location and, for each labeled cancer sample, the analytics system determines a count of cancer fragments terminating at each predictively significant location. The counts are encoded as feature values in a feature vector for each sample that is input to the classifier. Accordingly, based on the known label assigned to each sample and the encoded feature vector for each sample, which includes the count of fragments terminating at each predictively significant location, the cancer classifier is trained to output a cancer prediction.

FIG. 2B illustrates an example identification of predictively significant genomic locations, according to one embodiment. As an example, an analytics system receives a sequencing library 250 with sequence reads for a combination of labeled cancer cfDNA fragments and non-cancer cfDNA fragments. For the sake of simplicity, the illustrated fragments in the library 210 are between two and nine base pairs long. However, in practical wet lab implementations, cancer and non-cancer fragments analyzed by the analytics systems are between 50 or less and 140 or more base pairs.

In FIG. 2B, cancer fragments are illustrated with cross-hatched start points and end points and non-cancer fragments are illustrated with shaded start points and end points. The start point and end point of each DNA fragment are aligned to a base pair location on a reference genome. For the sake of simplicity, the illustrated sequencing library illustrates 14 base pair locations. However, the reference genome may include upwards of 3 billion base pair locations. At each of the 14 genomic locations, the analytics system determines a count of cancer fragments with end points at the location and updates an entry in the cancer endpoint position vector 230 corresponding to the location with the determined count. For example, three cancer fragments terminate at P₁₀, that is three cancer fragments have end points at P₁₀. Accordingly, the corresponding entry in the cancer endpoint position vector 230 is updated with a value of “3.” Similarly, the analytics system determines a count of non-cancer fragments with end points at each genomic location and updates a corresponding entry in a non-cancer endpoint position vector 220.

For each location on the reference genome, the analytics system performs a combination of statistical analyses to determine a p-value describing a likelihood that an elevated count of DNA fragment endpoints at the location is predictively significant in determining whether or not a subject has cancer. The p-value is determined as a computational comparison between the count of cancer fragments (as entered in the cancer endpoint position vector 230) and the count of non-cancer fragments (as entered in the non-cancer endpoint position vector 220). For example, the p-value determined for P₁₀ is determined to be 0.2 based on the elevated count of cancer fragments end points (3) relative to count of non-cancer fragment end points (1). In one embodiment, the array of p-values determined for locations on the reference genome is stored as a feature vector, for example the position p-value vector 250. In alternate embodiments, the array of p-values is stored by the analytics system as an alternate data structure.

The analytics system compares the determined p-value of each genomic location to a p-value threshold to identity predictively significant genomic locations 250. The p-value threshold may be tuned over time with a continuously updated dataset to confirm consistent accuracy of the model. In one embodiment, p-values below the p-value threshold are determined to be predictively significant. Accordingly, in the illustrated embodiment of FIG. 2B, in response to a comparison against a p-value threshold of 0.3, location P₁₀ (with a p-value of 0.2) is identified as a predictively significant genomic location 250.

II.B.I. P-Value Computation

In one embodiment, the analytics system calculates a p-value for each genomic location based on a plurality of samples including cancer fragments and non-cancer fragments. The analytics system compares a number of cancer fragments with endpoints at a genomic location (encoded as an entry in a cancer endpoint position vector) with a number of non-cancer fragments with endpoints at a genomic location (encoded as an entry in a cancer endpoint position vector). The p-value assigned to each location represents the likelihood that fragments are more likely to terminate at that position in cancer-derived cfDNA compared to non-cancer-derived cfDNA. In order to determine that a genomic location is predictively significant, the analytics system uses non-cancer samples from a healthy control group and generates a non-cancer endpoint position vector.

The analytics system implements a hierarchical Bayes approach to model an uncertainty in the rate of non-cancer fragments present at a location on a reference genome. The likelihood of non-cancer fragments terminating at each genomic location is modeled as a Poisson distribution. Described differently, the Poisson distribution for a genomic location models a likelihood that a count of non-cancer fragments be observed at the location over multiple, different test samples. For a given non-cancer sample, the count of fragment endpoints at each location may be a categorized as one of multiple different scenarios, for example a first low likelihood scenario that a count is significantly above the expected average count for the location, a second low likelihood scenario that a count is significantly below the expected average count for the location, and a high likelihood scenario that a count is approximately equivalent to the expected average count for the location. The Poisson distribution is generated as function of a rate parameter (k). Accordingly, a genomic location corresponding to an elevated count of non-cancer fragment endpoints relative to a second genomic location may result in an increase in λ relative to another genomic location with an average count of non-cancer fragment endpoints.

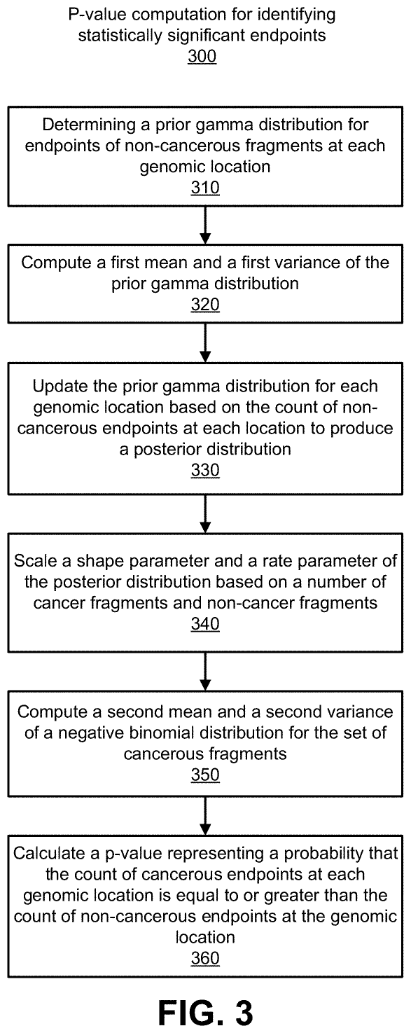

FIG. 3 is a flowchart describing a process 300 of computing a p-value for the identification of predictively significant genomic locations, according to one embodiment. The analytics system determines 310 a gamma distribution for endpoints of a non-cancer fragment at each genomic location, or alternatively a corresponding entry in the non-cancer endpoint position vector, that is a conjugate prior distribution of the Poisson distribution generated for the location. The analytics system models the Poisson rate parameter (λ) at each genomic location as a random variable drawn from the Gamma distribution. The Gamma prior distribution is parameterized by a shape parameter (α) and a rate parameter (β). Because of the a priori assumption that cfDNA fragments are equally likely to terminate at any position, the α and β parameters are estimated from pooled counts across genomic locations to generate a Gamma prior that is used for all positions. Accordingly, the analytics system aggregates endpoint counts for non-cancer fragments at each genomic location of a reference genome to fit the prior gamma distribution. The resulting Poisson distribution with a Gamma prior is then used to represent the count of non-cancer fragments for a genomic location on the reference genome. Because, for each genomic location, the counts from a Poisson distribution with a Gamma prior on λ follow a negative binomial distribution, the analytics system computes the mean and variance of the negative binomial distribution, which sufficiently parameterize the distribution. The mean and the variance may be computed according to Equation (1) and Equation (2), respectively.

$$\begin{matrix}μ = \frac{α}{β} & ( 1 ) \\ θ = \frac{α}{{β}^{2}} & ( 2 )\end{matrix}$$

In implementation, the analytics system fits a number of non-cancer fragments with endpoints at each genomic location to a Poisson distribution with a Gamma prior where the a α and β parameters share a prior distribution with corresponding α and β parameters from each of the other genomic locations on a reference genome. Accordingly, the α and β parameters may be used to characterize an expected number of non-cancer fragments terminating at a genomic location, in addition to describing how variable that count is between different genomic locations. Given the large number of genomic locations on a reference genome, for example ranging up to 3 billion locations, it may be impractical to simultaneously estimate a Gamma prior distribution for each genomic location using a hierarchical Bayesian model. In one embodiment, to optimize the process, the analytics system identifies a random subset of locations and fits a Gamma distribution to those locations. The analytics system may determine an estimate of a Gamma prior distribution shared by each location on a reference genome, for example using a mean estimate of the shared α and β parameters. The estimated Gamma prior distribution may be updated and applied across the reference genome on a location-by-location basis using the observed count of non-cancer fragments with endpoints at each location. As a result, the computation of the Gamma prior distribution for each location is informed by the counts at all other locations.

As a simplified example, for a given non-cancer sample, an endpoint count for one genomic locations is 0 and an endpoint count for another genomic location is 10. In a naïve implementation in which the Gamma prior distribution for each location is not informed by the Gamma prior distribution for other locations, the λ parameter for the Poisson distribution for a location is estimated based on the observed counts of non-cancer endpoints at the location. As a result, the Poisson distribution of the location with an endpoint count of 0 would have a λ of 0 and any location with a non-zero count in a cancer fragment would be considered infinitely significant. To overcome the flaw of such a naïve implementation, the analytics system uses the Bayesian setup described above (e.g., modeling endpoint counts for each genomic location as a Poisson distribution with a Gamma prior). The λ parameter for the location with an endpoint count of 0 is modeled as a Poisson distribution with a non-zero λ determined, to some extent, based on the non-zero λ value of the location with an endpoint count of 10. Using the implementation described above, a prior Gamma distribution may be determined that applies to each genomic location on a reference genome using an endpoint analysis of DNA fragments from a set of non-cancer samples.

As described above, based on analysis of a sequencing library generated from a set of non-cancer samples, the analytics system determines an actual count of non-cancer fragments with endpoints at each genomic location on a reference genome. The determined count for each location may be treated as an isolated observation and used to update 330 a previously generated prior distribution. Consistent with the description above, the prior distribution (e.g., the prior Gamma distribution) may be determined by fitting a control non-cancer sample for a genomic location. The analytics system updates the prior gamma distribution based on the count of sequenced non-cancer samples with endpoints at the genomic location to produce a posterior distribution of expected non-cancer end point counts at the location. Described differently, the analytics system may access the non-cancer endpoint position vector and update the prior gamma distribution for each genomic location based on the count recorded at a corresponding vector entry. The statistical analyses described above generate an updated expectation of the rate of non-cancer fragments at each genomic location in view of actual non-cancer endpoint counts from the observed event (i.e., the sequenced sample). The posterior distribution rate of non-cancer fragments terminating at each genomic location may be modeled according to Equation (3):

Pₚₒₛₜₑᵣᵢₒᵣ(λ)˜Gamma(α+x_{NC},β+1) (3),

where NC represents the actual count of non-cancer fragment endpoints observed in the isolated set. In such an implementation, the posterior distribution on lambda for each genomic location is parameterized as a Gamma distribution with a posterior shape parameter and a posterior rate parameter. The posterior shape parameter and the posterior rate parameter may be characterized by Equations (4) and (5), respectively.

αₚₒₛₜₑᵣᵢₒᵣ=α+x_{NC} (4)

βₚₒₛₜₑᵣᵢₒᵣ=β+1 (5)

For example, if no non-cancer fragment endpoints are observed at a genomic location, the analytics system determines a distribution (λ) indicating a lower likelihood that a DNA fragment will terminate at that location. In comparison, if 100 non-cancer fragment endpoints are observed at another genomic location, the analytics system determines a distribution (λ) indicating a higher likelihood that a DNA fragment will terminate at that location and that the number of DNA fragments terminating at that location may be more variable than other locations (e.g., it may be that in the cell type predominantly contributing cfDNA fragments, the nucleases that chew back the DNA tend to stop at that position). By applying non-cancer endpoints counts for a genomic location from an isolated observation to the prior Gamma distribution, the analytics system is able to tailor the expected counts of non-cancer endpoints for each location to the specific cfDNA fragments sequenced from the sample representing the isolated observation. The resulting posterior distribution for a given genomic location represents a location-specific rate of non-cancer counts at the given location

Biologically, the rate at which cancer and non-cancer fragments are observed at a genomic location is based on the rate at which tumor and non-cancer tissues shed fragments into circulating blood. Statistically, the Poisson distribution with a Gamma prior suggests that locations along a reference genome ought to behave similarly, so although a non-cancer endpoint count for a first location may be 0, if there are many other locations with low, but non-zero endpoint counts, the analytics system recognizes that the first location more likely has a low, non-zero endpoint count, rather than a 0 count.

Based on the position-specific rate of non-cancer endpoint counts at a given location, the analytics system can determine whether a number of cancer fragments terminating at a genomic location is abnormal enough to have any predictive significance in the classification or identification of cancer in a subject. From the generated sequencing library, the analytics system determines a count of cancer fragments terminating at each genomic location on the reference genome and stores the endpoint count for each location, for example in the form of a cancer endpoint position vector. For each location, the count of cancer fragment endpoints is treated as a new observation drawn from a Poisson distribution (λ) where λ is Gamma-distributed according to the posterior distribution described above. Given the outlined conditions, the probability of a cancer fragment terminating at a genomic location or the probability of a different, new observation may be modeled as a negative binomial distribution characterized by a mean and a variance consistent with Equations (6) and (7).

$$\begin{matrix}{μ}_{N e g B i n o m} = \frac{{α}_{P o s t e r i o r}}{{β}_{p o s t e r i o r}} & ( 6 ) \\ {θ}_{N e g B i n o m} = {α}_{Posterior} ( \frac{{β}_{p o s t e r i o r}^{+ 1}}{{β}_{p o s t e r i o r}^{2}} ) & ( 7 )\end{matrix}$$

In some implementations, the analytics system performs an additional statistical computation to scale 340 the posterior shape and posterior rate parameters of the posterior distributed Gamma distribution based on the read depth indicating a total count of cancer fragment endpoints and a total count of non-cancer fragment endpoints for a sample. For example, if a sample includes 10 times as many non-cancer fragments as cancer fragments, the non-cancer endpoint count for a given location would be on average 10× higher than the corresponding cancer endpoint count. Accordingly, the posterior distribution is scaled by a factor of 10 before determining the cancer endpoint count for a location and, then, the p-value of cancer endpoints at the location is determined from the scaled posterior distribution. Such a scaling computation is determined based on a combination of one or more of: known properties of the Gamma distribution, a ratio of total cancer endpoint counts on a reference genome to total non-cancer endpoint counts on the same reference genome, the scaled posterior shape and rate parameters, and a scaled mean and variance of negative binomial distribution. The scaled posterior shape and rate parameters are then used in Equations (4) and (5) to compute 350 an updated mean and variance of the negative binomial distribution, scaled based on the total count of non-cancer fragment endpoints relative to the total count of cancer fragment endpoints.

Given that the observation of cancer fragments endpoints at a genomic location follows a negative binomial distribution, the analytics system calculates 360 a p-value for the genomic location based on the updated mean and variance determined from the scaled negative binomial distribution for the location. Consistent with the description above, the p-value represents a probability that the rate of observing cancer fragments terminating at a given location is higher than the rate of non-cancer fragments terminating at the same location. The p-value of a particular count of fragment endpoints is the probability of observing that count (x_{c}) or a greater count of endpoints in a non-cancer sample. For example, a low p-value (e.g., a p-value below 10⁻⁵) for a genomic location indicates a low probability of observing that number of endpoint counts in that location for a non-cancer sample and, because of that low probability, the endpoint count at the location is potentially indicative of a patient having a cancer. The p-value computation may be performed using any computational means or statistical software. In one embodiment, the p-value for a location is calculated using the negative binomial distribution.

In an alternate implementation, the analytics system uses the negative binomial probability mass function to compute a probability that the count of non-cancer endpoints for a genomic location is equal to a defined integer greater than or equal to the count of endpoints within a corresponding entry in the non-cancer endpoint position vector. The analytics system may sum the computed probabilities for all counts greater or equal to the observed count to determine a p-value for each genomic location. In an alternate implementation, the analytics system uses the cumulative distribution function value for the observed count to determine the p-value for each genomic location.

The final computational result is a p-value of observed cancer fragment endpoints for each of the approximately 3 billion genomic locations on a human chromosome that reflects how likely it is that cancer fragments are more likely to terminate at that particular position than non-cancer fragments. In one implementation, a p-value closer to 0 represents an increased likelihood that cancer-derived fragments preferentially terminate at that position. As will be described below with reference to Section II.B.II, any p-value threshold then defines a set of positions that are enriched for cancer-derived fragments at least at a predictively significant level.

II.B.II. Predictively Significant Genomic Locations

The analytics system identifies predictively significant genomic locations with p-values under a p-value threshold. The analytics system implements a p-value filter that compares the p-value of each genomic location to the p-value threshold and identifies any locations assigned p-values over the threshold a predictively insignificant. P-values determined for each genomic location range between 0 and 1. P-values nearing 1 represent a lessened probability of cancer fragments occurring at the corresponding location, whereas p-values nearing 0 represent an increased probability of cancer fragments occurring at the corresponding location. Example p-value thresholds for identifying predictively significant locations include p-values of, or below, 10⁻¹⁰, 10⁻¹², 10⁻¹³, 10⁻¹⁴, 10⁻¹⁵, 10⁻¹⁶, 10⁻¹⁸, and 10⁻²⁰. In alternate embodiments, the p-value threshold may be less than one of the following: 10⁻², 10⁻³, 10⁻⁴, 10⁻⁵, 10⁻⁶, 10⁻⁷, 10⁻⁸, 10⁻⁹, 10⁻¹⁰, 10⁻¹¹, and 10⁻¹². lower than 10⁻⁹, lower than 10⁻¹⁰, lower than 10⁻¹¹, or lower than 10⁻¹². P-value thresholds may be defined based on considerations of the available training data, for example a number of available training samples, a sequencing depth of each sample, and a size range of the fragments being considered.

In some embodiments, the analytics system may implement multiple p-value thresholds against which p-values for a genomic location may be compared. In doing so, the analytics system generates tiers of predictively significant genomic locations and may train a cancer classifier to generate cancer predictions based on endpoint counts for a combination of locations in different tiers. Alternatively, the cancer classifier may be trained to generate a first cancer prediction based on endpoint counts of genomic locations in the first tier and a second cancer prediction based on endpoints counts of genomic locations in the second tier. For example, an analytics system may implement two p-value thresholds, T₁ and T₂, where T₁ is lower than T₂. Accordingly, a first tier of genomic locations with p-values below T₁ may be encoded as features to train a cancer classifier to generate cancer predictions with greater confidence than a comparable cancer classifier trained on identified locations between T₁ and T₂.

In some embodiments, in addition to the p-value filter, the analytics system implements a signal-to-noise filter to further identify predictively significant genomic locations for consideration. For each location, the signal to noise filter treats a count of cancer fragments with endpoints at the location as a signal and a count of non-cancer fragments with endpoints at the location as noise. For example, if a set of samples includes cancer samples that in aggregate have 10,000 endpoints at a particular location and non-cancer samples that in aggregate have 500 endpoints, the position would be considered statistically significant, but may not be considered predictively significant because of the poor signal to noise properties. Additionally, the analytics system may group locations based on biological features that are expected to correlate with noise rates (e.g. proximity to a transcription factor binding site) as these prior distributions may better reflect the true biology and lead to a better set of significant sites.

### II.C. Example Analytics System

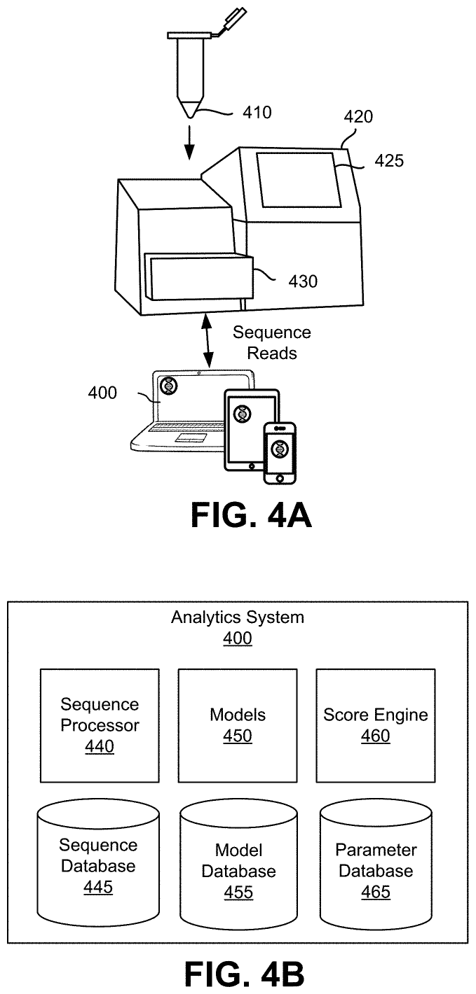

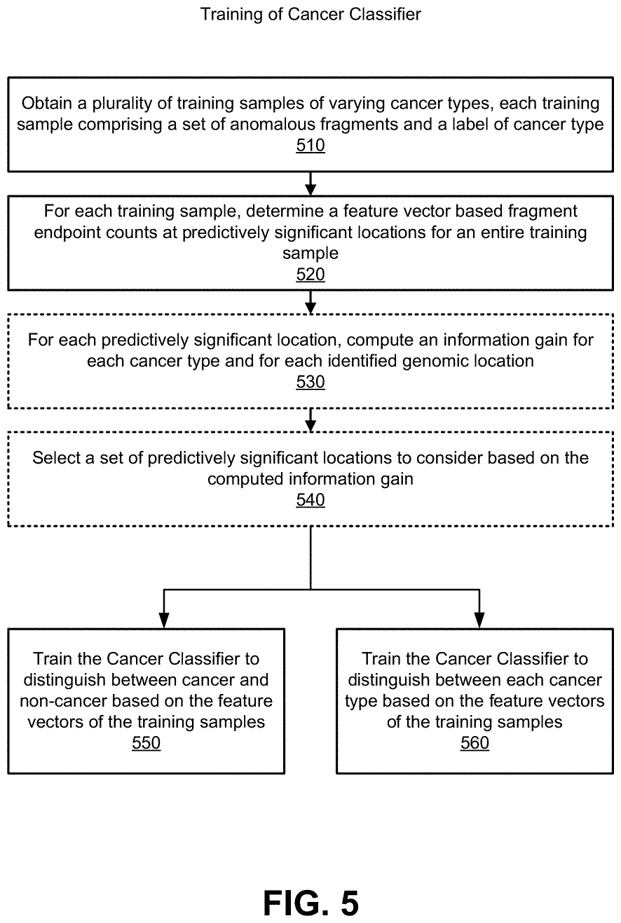

FIG. 4A is a flowchart of devices for sequencing nucleic acid samples according to one embodiment. This illustrative flowchart includes devices such as a sequencer 420 and an analytics system 400. The sequencer 420 and the analytics system 400 may work in tandem to perform one or more steps in the processes illustrated in FIGS. 1, 2A, 2B, 3, and other processes described herein.

In various embodiments, the sequencer 420 receives an enriched nucleic acid sample 410. As shown in FIG. 4A, the sequencer 420 can include a graphical user interface 425 that enables user interactions with particular tasks (e.g., initiate sequencing or terminate sequencing) as well as one more loading stations 430 for loading a sequencing cartridge including the enriched fragment samples and/or for loading necessary buffers for performing the sequencing assays. Therefore, once a user of the sequencer 420 has provided the necessary reagents and sequencing cartridge to the loading station 430 of the sequencer 420, the user can initiate sequencing by interacting with the graphical user interface 425 of the sequencer 420. Once initiated, the sequencer 420 performs the sequencing and outputs the sequence reads of the enriched fragments from the nucleic acid sample 410.

In some embodiments, the sequencer 420 is communicatively coupled with the analytics system 400. The analytics system 400 includes some number of computing devices used for processing the sequence reads for various applications such as identifying counts of DNA fragments with endpoints at locations on a reference genome. The sequencer 420 may provide the sequence reads in a BAM file format to the analytics system 400. The analytics system 400 can be communicatively coupled to the sequencer 420 through a wireless, wired, or a combination of wireless and wired communication technologies. Generally, the analytics system 400 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to identify the endpoints of each cfDNA fragment, e.g., via step 120 of the process illustrated in FIG. 1A. Endpoint analysis may generally describe the identification of the location on the refence genome where each cfDNA fragment (cancer-type or non-cancer type) terminates. For each reference genomic location, a count of non-cancer fragments terminating at the location and a count of cancer fragments terminating at the location are analyzed to determine whether the location is predictively significant. Based on locations identified as predictively significant, a classifier is trained to make a determination regarding whether a subject has cancer, and in some embodiments, the type of cancer.

A region in the reference genome may be associated with a gene or a fragment of a gene; as such, the analytics system 400 may label a sequence read with one or more genes that align to the sequence read. In one embodiment, fragment length (or size) is determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Information derived from an endpoint analysis of the read pair R_1 and R_2 may include a start position in the reference genome that corresponds to an end of a first read (e.g., R_1) and an end position in the reference genome that corresponds to an end of a second read (e.g., R_2). In other words, the start position and end position in the reference genome represent the likely location within the reference genome to which the nucleic acid fragment corresponds. An output file having SAM (sequence alignment map) format or BAM (binary) format may be generated and output for further analysis.

Referring now to FIG. 4B, FIG. 4B is a block diagram of an analytics system 400 for processing DNA samples according to one embodiment. The analytics system implements one or more computing devices for use in analyzing DNA samples. The analytics system 400 includes a fragment processor 440, sequence database 445, modes 450, model database 455, score engine 460, and parameter database 465. In some embodiments, the analytics system 400 performs some or all of the processes 100 of FIG. 1A and 200 of FIG. 2.

The sequence processor 440 generates endpoint position vector for fragments from a sample. In one embodiment, the sequence processor 440 generates a count of non-cancer fragments terminating at each location on a reference genome and a count of cancer fragments terminating at the location. The sequence processor 440 generates a non-cancer endpoint position vector where each entry of the vector corresponds to a location on the reference genome and is populated with the count of non-cancer fragments terminating at the location. Similarly, the processor 440 generates a cancer endpoint position vector where each entry is populated with a count of cancer fragments terminating at the corresponding genomic location. The sequence processor 440 may store the pair of endpoint position vectors for a sample in the sequence database 445. Data in the sequence database 445 may be organized such that the endpoint position vectors from a sample are related to one another.

Further, multiple different models 450 may be stored in the model database 455 or retrieved for use with test samples. In one example, a model is a trained cancer classifier for determining a cancer prediction for a test sample using a feature vector derived from cfDNA fragments of the sample. The training and use of the cancer classifier will be further discussed in conjunction with Section III. Cancer Classifier for Determining Cancer. The analytics system 400 may train the one or more models 450 and store various trained parameters in the parameter database 465. The analytics system 400 stores the models 450 along with functions in the model database 455.

During inference, the score engine 460 uses the one or more models 450 to return outputs. The score engine 460 accesses the models 450 in the model database 455 along with trained parameters from the parameter database 465. According to each model, the score engine receives an appropriate input for the model and calculates an output based on the received input, the parameters, and a function of each model relating the input and the output. In some use cases, the score engine 460 further calculates metrics correlating to a confidence in the calculated outputs from the model. In other use cases, the score engine 460 calculates other intermediary values for use in the model.

### III. Cancer Classifier for Determining Cancer

III.A. Overview

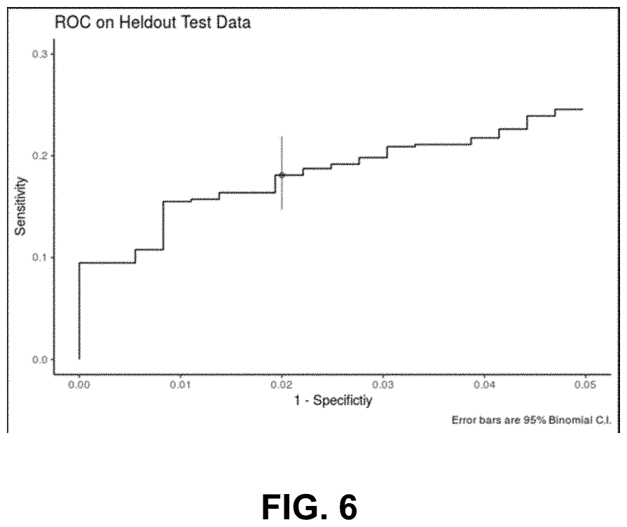

The cancer classifier is trained to receive a feature vector for a test sample and determine whether the test sample is from a test subject that has cancer or, more specifically, a particular cancer type. The cancer classifier comprises a plurality of classification parameters and a function representing a relation between the feature vector as input and the cancer prediction as output determined by the function operating on the input feature vector with the classification parameters. In one embodiment, the feature vectors input into the cancer classifier are based on a set of cfDNA fragments determined from the test sample. For each sample, the feature vector input to the classifier includes a count of fragment endpoints that appear at each predictively significant location on the reference genome. The significant locations may be determined by the processes described in FIGS. 1-3 before being encoded into feature values included in the feature vector. Prior to the deployment of the cancer classifier, the analytics system trains the cancer classifier, the process illustrated in FIG. 5.

III.B. Training of Cancer Classifier

FIG. 5 is a flowchart describing a process of training a cancer classifier, according to an embodiment. The analytics system obtains 510 a plurality of training samples each having a set of sequenced fragments aligned to a reference genome and a label of cancer type. The plurality of training samples includes any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

The analytics system determines 520, for each training sample, a feature vector based on a set of classifier parameters and fragment endpoint counts at predictively significant locations for the entire training sample. The analytics system identifies predictively significant locations based on the p-value computation described above for a set of labeled cancer and non-cancer segments from a sample. The locations encoded into a feature vector for a training sample may include all locations identified as predictively significant, for example any locations with a p-value below a threshold. Once all predictively significant endpoint counts for fragments of a test sample are determined, the analytics system determines the feature vector as vector elements including the endpoint count at each identified location. In one embodiment, the cancer classifier is trained using high-tumor fraction (>5%) training sample(s).

In one embodiment, the analytics system computes 530 an information gain for each cancer type and for each identified genomic location to determine whether to include the location in the classifier. The information gain is computed for training samples with a given cancer type compared to all other samples. For example, each sample in a training set may be characterized by random variables including ‘endpoint count’ (‘EC’) for each predictively significant location and ‘cancer type’ (‘CT’). In one embodiment, EC is a numeric variable quantifying the count of fragment endpoints detected at a given location on the reference genome. CT is a random variable indicating whether the cancer is of a particular type. Alternatively, CT is a binary variable indicating whether a sample is a cancer-type or a non-cancer type. The analytics system computes the mutual information with respect to CT given the combination of EC values. That is, how many bits of information about the cancer type are gained if the counts of endpoint fragments at each predictively significant location are known.

For a given cancer type, the analytics system uses this information to rank predictively significant locations based on how cancer specific they are. This procedure is repeated for all cancer types under consideration. If a particular genomic location is commonly associated with an uncharacteristically high number of fragment endpoints in training samples of a given cancer but not in training samples of other cancer types or in healthy training samples, then samples with elevated endpoint counts at that location will tend to have high information gains for the given cancer type. The ranked predictively significant locations for each cancer type are added (selected) 340 to a selected set of genomic locations based on their rank for use in the cancer classifier.

With the feature vectors of the training samples, the analytics system may train the cancer classifier in any of a number of ways. The feature vectors may correspond to the initial set of genomic locations identified relative to the p-value threshold from step 520 or to the selected set of locations from step 540. In one embodiment, the analytics system trains 550 a binary cancer classifier to distinguish between cancer and non-cancer based on the feature vectors of the training samples. In this manner, the analytics system uses training samples that include both non-cancer samples from healthy individuals and cancer samples from subjects. Each training sample has one of the two labels “cancer” or “non-cancer.” In this embodiment, the classifier outputs a cancer prediction indicating the likelihood of the presence or absence of cancer.

In another embodiment, the analytics system trains 560 a multiclass cancer classifier, to distinguish between many cancer types. Cancer types include one or more cancers and may include a non-cancer type (may also include any additional other diseases or genetic disorders, etc.). To do so, the analytics system uses the cancer type cohorts and may also include or not include a non-cancer type cohort. In this multi-cancer embodiment, the cancer classifier is trained to determine a cancer prediction that comprises a prediction value for each of the cancer types being classified. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further process the prediction values to generate a single cancer determination. Continuing with the example above and given the percentages, in this example the system may determine that the sample has breast cancer given that breast cancer has the highest likelihood.

As an alternative to the multiclass classifier, the analytics system may train different cancer classifiers to generate a cancer prediction for a particular type of cancer. Accordingly, each classifier may be trained on training samples derived from subjects with the particular type of cancer. In such implementations, the analytics system implements the endpoint analysis processes described above to identify predictively significant genomic locations for each type of cancer.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier is sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

III.C. Deployment of Cancer Classifier

During use of the cancer classifier, the analytics system obtains a test sample from a subject of unknown cancer type. The analytics system may process the test sample comprising DNA molecules with any combination of processes described above to identify predictively significant genomic locations and a count of DNA fragments terminating at each identified location. The analytics system encodes the count of fragments identified at each predictively significant location into a feature value included in a feature vector for use by the cancer classifier as described above. For example, the cancer classifier receives as input, test feature vectors inclusive of endpoint counts for several genomic locations from several test samples with p-values below a p-value threshold.

The analytics system then inputs the test feature vector into the cancer classifier. The function of the cancer classifier then generates a cancer prediction based on the classification parameters trained in the process 300 and the test feature vector. In the first manner, the cancer prediction is binary and selected from a group consisting of “cancer” or non-cancer;” in the second manner, the cancer prediction is selected from a group of many cancer types and “non-cancer.” In additional embodiments, the cancer prediction has prediction values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains a cancer classifier trained in step 550 of FIG. 5 with another cancer classifier trained in step 560. The analytics system inputs the test feature vector into the cancer classifier trained as a binary classifier in step 550. The analytics system receives an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier receives the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

### IV. Cancer Assay Panel

IV.A. Deployment of Cancer Assay Panel

In one aspect, the present description provides a cancer assay panel (e.g., a bait set) comprising a plurality of probes or a plurality of probe pairs. The probes can be polynucleotide-containing probes that are specifically designed to target one or more nucleic acid molecules corresponding to, or derived from genomic regions having fragment endpoints useful as markers for distinguishing between cancer and non-cancer samples, between different cancer tissue of origin types, between different cancer cell types, or between samples of different stages of cancer, as identified by methods provided herein. In some embodiments, probes target genomic regions (or nucleic acid molecules derived therefrom) having fragment endpoint patterns specific to one or more cancer types, including, for example, (1) blood cancer, (2) breast cancer, (3) colorectal cancer, (4) esophageal cancer, (5) head and neck cancer, (6) hepatobiliary cancer, (7) lung cancer, (8) ovarian cancer, or (9) pancreatic cancer. In some embodiments, the panel includes probes targeting genomic regions specific to a single cancer type. In some embodiments, the panel includes probes specific to 2, 3, 4, 5, 6, 7, 8, or 9 or more cancer types. In some embodiments, the target genomic regions having informative endpoint locations are selected to maximize classification accuracy, subject to a size limitation (which can be determined by a sequencing budget and a desired depth of sequencing).

For designing the cancer assay panel, an analytics system may collect samples corresponding to various outcomes under consideration, e.g., samples known to have cancer, samples considered to be healthy, samples from a known tissue of origin, etc. The analytics system may be any generic computing system with a computer processor and a computer-readable storage medium with instructions for execution by the computer processor to perform any or all operations described in this present disclosure. With the samples, the analytics system determines endpoint positions for each nucleic acid fragment in the sample. The analytics system may then select target genomic regions based on fragment endpoint patterns. From the selected target genomic regions with high distinguishability power, the analytics system may design probes to target nucleic acid fragments inclusive of the selected genomic endpoint positions. The analytics system may generate variable sizes of the cancer assay panel, e.g., where a small sized cancer assay panel includes probes targeting the most informative genomic region, a medium sized cancer assay panel includes probes from the small sized cancer assay panel and additional probes targeting a second tier of genomic regions with informative endpoints, and a large sized cancer assay panel includes probes from the small sized and the medium sized cancer assay panels and even more probes targeting a third tier of informative genomic regions. With such cancer assay panels, the analytics system may train classifiers with various classification techniques to predict a sample's likelihood of having a particular outcome, e.g., cancer, specific cancer type, other disorder, etc.

Specifically, in some embodiments, the cancer assay panel comprises a plurality of different polynucleotide probes configured to hybridize to a nucleotide sequence of a polynucleotide having an informative genomic endpoint position. For example, each of the different polynucleotide probes can be configured to hybridize to, and enrich, a plurality of nucleotide sequences having a genomic endpoint listed in any one of Tables I-IV.

In some other embodiments, the cancer assay panel comprises at least 50 pairs of probes, wherein each pair of the at least 50 pairs comprises two probes configured to overlap each other by an overlapping sequence, wherein the overlapping sequence comprises a 30-nucleotide sequence, and wherein each of the two different probes of each of the pair of probes is configured to hybridize to a nucleotide sequence of a polynucleotide having a genomic endpoint. In other embodiments, the cancer assay panel can comprise at least 60, 70, 80, 90, 100, 200, 300, 500, 1,000, 1,500, or 2,000 pairs of probes.

In some embodiments, probes are conjugated to a tag (e.g., a non-nucleic acid affinity moiety), such as a biotin moiety.

Each of the probes (or probe pairs) can be designed to target nucleic acids derived from one or more target genomic regions with informative endpoints. The target genomic regions having informative endpoints are selected based on several criteria designed to increase selective enrichment of informative cfDNA fragments while decreasing noise and non-specific bindings.

In one example, a panel can include probes that can selectively hybridize to (i.e., bind to) and enrich cfDNA fragments that have fragment endpoints that are more prevalent, and thus, informative in cancerous samples. In this case, sequencing of the enriched fragments can provide information relevant to the detection, diagnosis, and/or prognosis of cancer.

Each of the probes can target a genomic region comprising at least 30 bp, 35 bp, 40 bp, 45 bp, 50 bp, 60 bp, 70 bp, 80 bp, 90 bp, 100 bp or more.

Further filtration can be performed to select probes with high specificity for enrichment (i.e., high binding efficiency) of nucleic acids derived from targeted genomic endpoint locations. Probes can be filtered to reduce non-specific binding (or off-target binding) to nucleic acids derived from non-targeted genomic endpoint locations. For example, probes can be filtered to select only those probes having less than a set threshold of off-target binding events. In one embodiment, probes can be aligned to a reference genome (e.g., a human reference genome) to select probes that align to less than a set threshold of regions across the genome. For example, probes can be selected that align to less than 25, 24, 23, 22, 21, 20, 19, 18, 17, 16, 15, 14, 13, 12, 11, 10, 9 or 8 off-target locations across the reference genome. In other cases, filtration is performed to remove genomic regions when the sequence of the target genomic endpoint location appears more than 5, 10, 15, 20, 21, 22, 23, 24, 25, 26, 27, 28, 29, 30, 31, 32, 33, 34 or 35 times in a genome. This is for excluding probes that can pull down off-target fragments, which are not desired and can impact assay efficiency.

Once the probes hybridize and capture DNA fragments corresponding to, or derived from, a target genomic region having an informative endpoint location, the hybridized probe-DNA fragment intermediates are pulled down (or isolated), and the targeted DNA is amplified and sequenced. The sequence read provides information relevant for diagnosis of cancer. For this end, a panel is designed to include a plurality of probes that can capture fragments that can together provide information relevant to diagnosis of cancer. In some embodiments, a panel includes at least 50, 60, 70, 80, 90, 100, 200, 300, 500, 1,000, or different polynucleotide probes. In other embodiments, a panel includes at least at least 50, 60, 70, 80, 90, 100, 120, 150, 200, 500, 1,000, or 2,000 different pairs of probes, wherein each pair of the at least 50, 60, 70, 80, 90, 100, 120, 150, 200, 500, 1,000, or 2,000 different pairs of probes comprises two different probes configured to overlap with each other by an overlapping sequence of at least 30 or more contiguous nucleotides and wherein each of the two different probes of each of the pair of probes is configured to hybridize to a nucleotide sequence of a polynucleotide having a genomic endpoint. In other embodiments, the overlapping sequence can be at least 40, 50, 60, 70, or 80 contiguous nucleotides. The plurality of probes together can comprise at least 0.01 million, 0.02 million, 0.03 million, 0.04 million, 0.05 million, 0.1 million, 0.2 million, 0.4 million, 0.6 million, 0.8 million, or 1 million.

IV.B. Enrichment Probes for Cancer Assay Panel

Cancer assay panels (e.g., bait sets) provided herein can include a set of hybridization probes (also referred to herein as “probes”) designed to, during enrichment, target and pull down (e.g., via hybridization capture) nucleic acid fragments of interest for the assay. In some embodiments, the probes are designed to hybridize and enrich a plurality of DNA molecules having informative endpoint positions for cancer, cancer type, or cancer tissue of origin. The target strand can be the “positive” strand (e.g., the strand transcribed into mRNA, and subsequently translated into a protein) or the complementary “negative” strand. In a particular embodiment, a cancer assay panel includes sets of two probes, one probe targeting the positive strand and the other probe targeting the negative strand of a target genomic region.

For each target genomic region having an informative endpoint location, four possible probe sequences can be designed. DNA molecules corresponding to, or derived from, each target region is double-stranded, as such, a probe or probe set can target either the “positive” or forward strand or its reverse complement (the “negative” strand). Additionally, in some embodiments, the probes or probe sets are designed to enrich DNA molecules or fragments that have been processed to convert unmethylated cytosines (C) to uracils (U). In accordance with this embodiment, because the probes or probe sets are designed to enrich DNA molecules corresponding to, or derived from the targeted regions with informative endpoint locations after conversion, the probe's sequence can be designed to enrich DNA molecules of fragments where unmethylated C's have been converted to U's (by utilizing A's in place of G's at sites that are unmethylated cytosines in DNA molecules or fragments corresponding to, or derived from, the targeted region). In one embodiment, probes are designed to bind to, or hybridize to, DNA molecules or fragments from genomic regions known to contain cancer-specific endpoint location patterns, thereby enriching for cancer-specific DNA molecules or fragments. Targeting genomic regions with cancer-specific endpoint location patterns, can be advantageous allowing one to specifically enrich for DNA molecules or fragments identified as informative for cancer or cancer tissue of origin, and thus, lowering sequencing needs and sequencing costs. In other embodiments, two probe sequences can be designed for each target genomic region with an informative endpoint location (one for each DNA strand).

The probes can range in length from 10s, 100s, 200s, or 300s of base pairs. The probes can comprise at least 45, 50, 75, 100, or 120 nucleotides. The probes can comprise less than 300, 250, 200, or 150 nucleotides. In an embodiment, the probes comprise 45-200 or 100-150 nucleotides. In one particular embodiment, the probes comprise 120 nucleotides.

The probes are designed to analyze endpoint locations of target genomic regions (e.g., of the human or another organism) that are suspected to correlate with the presence or absence of cancer generally, presence or absence of certain types of cancers, cancer stage, or presence or absence of other types of diseases.

Furthermore, the probes can be designed to effectively hybridize to (or bind to) and pull down cfDNA fragments containing a target genomic region. In some embodiments, the probes are designed to cover overlapping portions of a target genomic region, so that each probe is “tiled” in coverage such that each probe overlaps in coverage at least partially with another probe in the library. In such embodiments, the panel contains multiple pairs of probes, where each pair comprises at least two probes overlapping each other by an overlapping sequence of at least 25, 30, 35, 40, 45, 50, 60, 70, 75 or 100 nucleotides, and wherein each of the two different probes of each of the pair of probes is configured to hybridize to a nucleotide sequence of a polynucleotide having an informative genomic endpoint. In some embodiments, the overlapping sequence can be designed to have sequence homology with or to be complementary to a target genomic region (or a converted version thereof), thus a nucleotide fragment corresponding to or derived from, or containing the target genomic region having an informative endpoint location can be bound and pulled down by at least one of the probes.

In one embodiment, a 2× tiled design, where each base in a target endpoint location is overlapped by two probes. For instance, each pair of probes may include a first probe and a second probe that both differs from the first probe and overlaps in sequence with the first probe (e.g., overlap by at least 30 nucleotides). This is done to ensure that even relatively short DNA fragments (e.g., 100 bp) corresponding to, or derived from a targeted region having an informative endpoint location, are guaranteed to have a substantial overlap (or sequence complementarity) with at least one probe, allowing for efficient capture of the relatively short DNA fragment. For example, a 100-bp DNA fragment overlapping a 30 bp target region would have at least a 75 bp overlap with at least one of the two probes. Other levels of tiling are possible. For example, to increase target size and capture efficiency, more probes can be tilted over a given target genomic endpoint location. To increase capture of any DNA fragment that overlaps the target endpoint, the probes can be designed to extend past the ends of the target region having an informative endpoint location on either side on both sides. For example, probes can be designed to extend past the ends of a 30-bp target region by at least 50 bp, 60 bp, 70 bp, 80 bp, 90, or 100 bp.

In some embodiments, each of the one or more targeted genomic endpoint locations is selected from one or more of Tables I-IV. In some embodiments, each of the one or more genomic regions is selected from Table I. In some embodiments, each of the one or more genomic regions is selected from Table II. In some embodiments, each of the one or more genomic regions is selected from Table III. In some embodiments, each of the one or more genomic regions is selected from Table IV.

In some embodiments, an entirety of probes on the panel together are configured to hybridize to DNA fragments obtained from the cfDNA molecules corresponding to or derived from at least 30%, 40%, 50%, 60%, 70%, 80%, 90% or 95% of the genomic regions in one or more of Tables I-IV. In some embodiments, an entirety of probes on the panel together are configured to hybridize to DNA fragments obtained from the cfDNA molecules corresponding to or derived from at least 30%, 40%, 50%, 60%, 70%, 80%, 90% or 95% of the genomic regions in one or more of the genomic regions in Table I. In some embodiments, an entirety of probes on the panel together are configured to hybridize to DNA fragments obtained from the cfDNA molecules corresponding to or derived from at least 30%, 40%, 50%, 60%, 70%, 80%, 90% or 95% of the genomic regions in one or more of the genomic regions in Table II. In some embodiments, an entirety of probes on the panel together are configured to hybridize to DNA fragments obtained from the cfDNA molecules corresponding to or derived from at least 30%, 40%, 50%, 60%, 70%, 80%, 90% or 95% of the genomic regions in one or more of the genomic regions in Table III. In some embodiments, an entirety of probes on the panel together are configured to hybridize to DNA fragments obtained from the cfDNA molecules corresponding to or derived from at least 30%, 40%, 50%, 60%, 70%, 80%, 90% or 95% of the genomic regions in one or more of the genomic regions in Table IV.

In some embodiments, an entirety of probes on the panel together are configured to hybridize to DNA fragments obtained from the cfDNA molecules corresponding to or derived from at least 50, 60, 70, 80, 90, 100, 200, 300, 500, 1,000, 1,500, or 2,000 genomic endpoint locations in one or more of Tables I-IV. In some embodiments, an entirety of probes on the panel together are configured to hybridize to DNA fragments obtained from the cfDNA molecules corresponding to or derived from at least 50, 60, 70, 80, 90, 100, 200, 300, 500, 1,000, 1,500, or 2,000 genomic endpoint locations listed in Table I. In some embodiments, an entirety of probes on the panel together are configured to hybridize to modified fragments obtained from the cfDNA molecules corresponding to or derived from at least 50, 60, 70, 80, 90, 100, 200, 300, or 500, genomic endpoint locations listed in Table II. In some embodiments, an entirety of probes on the panel together are configured to hybridize to DNA fragments obtained from the cfDNA molecules corresponding to or derived from at least 50, or 100 genomic endpoint locations listed in Table III. In some embodiments, an entirety of probes on the panel together are configured to hybridize to DNA fragments obtained from the cfDNA molecules corresponding to or derived from at least 50 genomic endpoint locations listed in Table IV.

### V. Applications

In some embodiments, the methods, analytic systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment.

V.A. Early Detection of Cancer

In some embodiments, the methods and/or classifier of the present invention are used to detect the presence or absence of cancer in a subject suspected of having cancer. For example, a classifier (e.g., as described above in Section III and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e. binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematologic malignancies, including but not limited to non-Hodgkin's lymphoma (NHL), multiple myeloma and acute hematologic malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

V.B. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years.

V.C. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In other embodiments, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### VI. Example Results of Cancer Classifier

VI.A. Sample Collection and Processing

Study design and samples: CCGA (NCT02889978) is a prospective, multi-center, case-control, observational study with longitudinal follow-up. De-identified biospecimens were collected from approximately 15,000 participants from 142 sites. Samples were divided into training (1,785) and test (1,015) sets; samples were selected to ensure a prespecified distribution of cancer types and non-cancers across sites in each cohort, and cancer and non-cancer samples were frequency age-matched by gender.

VI.B. Empirical Cancer Classification

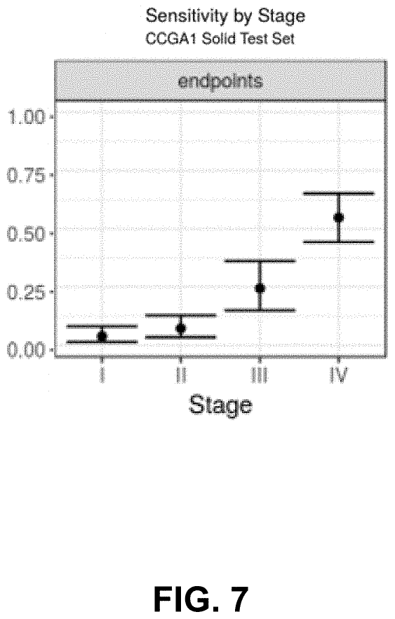

FIGS. 6-7 illustrates a graph showing a sensitivity by stage analysis of a cancer classifier, according to one embodiment. The cancer classifier used to produce the results shown in FIGS. 6-7 are trained according to example implementations of the process described above with reference to FIG. 5.

FIG. 6 illustrates a receiver operating characteristic curve illustrating the diagnostic ability of a binary cancer classifier using endpoint analysis, according to one embodiment. As illustrated by the curve, the classifier indicates an increase in true-positive detection rate with an accompanying increase in false-positive rate.

FIG. 7 illustrates a graph showing cancer prediction accuracy of a binary cancer classifier, according to an example implementation. In this illustrative example, the binary cancer classifier is trained to correctly identify a sample as cancer-type based on a count of endpoints observed at one or more predictively significant genomic locations. The classifier was evaluated against positive cancer samples from tissues at different stages of the disease. As is illustrated, the classifier exhibited a sensitivity between 0 and 0.25 when evaluated against samples with Stage I cancer. As expected, for samples in which the cancer had progressed from Stage I, the classifier exhibited increased sensitivity with notable improvements between analysis of Stage 3 and Stage 4 samples.

### VII. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

1. A method, comprising:
accessing a first set of training data and a second set of training data, the first set of training data indicating fragment endpoints of cell-free fragments from cancer samples and the second set of training data indicating fragment endpoints of cell-free fragments from non-cancer samples;
generating a first vector representative of the first set of training data and a second vector representative of the second set of training data, wherein each entry within the first vector and each entry within the second vector includes a count of cell-free fragments from the cancer samples and from the non-cancer samples, respectively, having an endpoint at a particular genomic location;
computing, for each genomic location, an associated p-value representative of a significance of the entry corresponding to the genomic location within the first vector relative to the entry corresponding to the genomic location within the second vector;
identifying a set of genomic locations associated with p-values less than a p-value threshold;
training a classifier based on the identified set of genomic locations; and
classifying a test sample as a cancer sample or a non-cancer sample by:
determining counts of cell-free fragments in the test sample having an endpoint at each of the identified genomic locations; and
applying the classifier to the determined counts to determine if the test sample is a cancer sample or a non-cancer sample.

2. The method of claim 1, wherein computing a p-value for each genomic location comprises:
determining a prior gamma distribution for the corresponding entry of the second vector, the prior gamma distribution parameterized by a prior shape parameter and a prior rate parameter used to compute a first mean of the prior gamma distribution and a first variance of the prior gamma distribution.

3. The method of claim 2, wherein computing a p-value for each genomic location further comprises:
updating the prior gamma distribution for the corresponding entry of the second vector based on the count of endpoints within the corresponding entry of the second vector to produce a posterior distribution of expected counts within the entry of the second vector, the posterior distribution parameterized by a posterior shape parameter and a posterior rate parameter.

4. The method of claim 3, wherein computing a p-value for each genomic location further comprises:
computing a second mean and a second variance of a negative binomial distribution for the entry of the first vector using the posterior shape parameter and the posterior rate parameter.

5. The method of claim 4, wherein computing the second mean and the second variance of the negative binomial distribution comprises scaling the posterior shape parameter and the posterior rate parameter based on a total count of cancer fragments from the cancer samples and a total count of non-cancer fragments from the non-cancer samples, and wherein the second mean and the second variance of the negative binomial distribution are computed based on the scaled posterior shape parameter and the scaled posterior rate parameter.

6. The method of claim 4, wherein computing a p-value for each genomic location further comprises:
computing, using the negative binomial distribution, a probability that a count of endpoints within an entry of the first vector corresponding to the genomic location is expected given a count of endpoints within an entry of the second vector corresponding to the genomic location or exceeds the count of endpoints within the entry of the second vector, wherein the computed probability comprises the p-value for the genomic location.

7. The method of claim 4, wherein computing a p-value for each genomic location further comprises:
computing, using the negative binomial probability mass function and for each integer greater than or equal to the count of endpoints within the entry of the first vector corresponding to the genomic location, a probability that the count of endpoints within the entry of the first vector corresponding to the genomic location is equal to the integer; and
summing the computed probabilities to produce the p-value.

8. The method of claim 1, wherein the cancer samples and the non-cancer samples comprise cell-free fragments between 50 and 140 bp.

9. The method of claim 1, wherein the cancer samples are from subjects with a particular type of cancer, and wherein the classifier is configured to classify the test sample as a cancer sample with the particular type of cancer or a non-cancer sample.

10. The method of claim 1, wherein the classifier is a multiclass classifier, and is configured to classify the test sample as a sample associated with one of a plurality of types of cancer.

11. The method of claim 1, further comprising:
identifying a second set of genomic locations associated with p-values greater than the p-value threshold but less than a second p-value threshold; and
training the classifier based additionally on the identified second set of genomic locations;
wherein classifying the test sample further comprises determining counts of cell-free fragments in the test sample having an endpoint at each of the second set of genomic locations.

12. The method of claim 1, wherein the p-value threshold is less than 10⁻⁴.

13. The method of claim 1, wherein the p-value threshold is one of: 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, 10⁻¹⁸, 10⁻¹⁹, and 10⁻²⁰.

14. The method of claim 1, wherein the set of genomic locations comprise less than 2,000, less than 5,000, less than 10,000, less than 50,000, less than 100,000, less than 500,000, less than 1 million, or less than 5 million unique genomic locations.

15.-44. (canceled)

45. A system comprising:
a computer processor; and
a non-transitory computer-readable storage medium storing instructions that, when executed by the computer processor, cause the computer processor to perform operations comprising:
accessing a first set of training data and a second set of training data, the first set of training data indicating fragment endpoints of cell-free fragments from cancer samples and the second set of training data indicating fragment endpoints of cell-free fragments from non-cancer samples;
generating a first vector representative of the first set of training data and a second vector representative of the second set of training data, wherein each entry within the first vector and each entry within the second vector includes a count of cell-free fragments from the cancer samples and from the non-cancer samples, respectively, having an endpoint at a particular genomic location;
computing, for each genomic location, an associated p-value representative of a significance of the entry corresponding to the genomic location within the first vector relative to the entry corresponding to the genomic location within the second vector;
identifying a set of genomic locations associated with p-values less than a p-value threshold;
training a classifier based on the identified set of genomic locations; and
classifying a test sample as a cancer sample or a non-cancer sample by:
determining counts of cell-free fragments in the test sample having an endpoint at each of the identified genomic locations; and
applying the classifier to the determined counts to determine if the test sample is a cancer sample or a non-cancer sample.

46. The system of claim 45, wherein computing a p-value for each genomic location comprises:
determining a prior gamma distribution for the corresponding entry of the second vector, the prior gamma distribution parameterized by a prior shape parameter and a prior rate parameter used to compute a first mean of the prior gamma distribution and a first variance of the prior gamma distribution.

47. The system of claim 46, wherein computing a p-value for each genomic location further comprises:
updating the prior gamma distribution for the corresponding entry of the second vector based on the count of endpoints within the corresponding entry of the second vector to produce a posterior distribution of expected counts within the entry of the second vector, the posterior distribution parameterized by a posterior shape parameter and a posterior rate parameter.

48. The system of claim 47, wherein computing a p-value for each genomic location further comprises:
computing a second mean and a second variance of a negative binomial distribution for the entry of the first vector using the posterior shape parameter and the posterior rate parameter.

49. The system of claim 48, wherein computing the second mean and the second variance of the negative binomial distribution comprises scaling the posterior shape parameter and the posterior rate parameter based on a total count of cancer fragments from the cancer samples and a total count of non-cancer fragments from the non-cancer samples, and wherein the second mean and the second variance of the negative binomial distribution are computed based on the scaled posterior shape parameter and the scaled posterior rate parameter.

50. A non-transitory computer-readable storage medium storing instructions that, when executed by a computer processor, cause the computer processor to perform operations comprising:
accessing a first set of training data and a second set of training data, the first set of training data indicating fragment endpoints of cell-free fragments from cancer samples and the second set of training data indicating fragment endpoints of cell-free fragments from non-cancer samples;
generating a first vector representative of the first set of training data and a second vector representative of the second set of training data, wherein each entry within the first vector and each entry within the second vector includes a count of cell-free fragments from the cancer samples and from the non-cancer samples, respectively, having an endpoint at a particular genomic location;
computing, for each genomic location, an associated p-value representative of a significance of the entry corresponding to the genomic location within the first vector relative to the entry corresponding to the genomic location within the second vector;
identifying a set of genomic locations associated with p-values less than a p-value threshold;
training a classifier based on the identified set of genomic locations; and
classifying a test sample as a cancer sample or a non-cancer sample by:
determining counts of cell-free fragments in the test sample having an endpoint at each of the identified genomic locations; and
applying the classifier to the determined counts to determine if the test sample is a cancer sample or a non-cancer sample.
